(19) United States
(12) Patent Application Publication
(10) Pub. No.: US 2025/0171832 A1
(43) Pub. Date: May 29, 2025

(54) BLOCKER METHODS

(71) Applicant: Illumina, Inc., San Diego, CA (US)

(72) Inventors: Claire Bevis-Mott, Cambridge (GB); Debra Sue Bryan, II, San Diego, CA (US)

(73) Assignee: Illumina, Inc., San Diego, CA (US)

(21) Appl. No.: 18/695,281

(22) PCT Filed: Sep. 30, 2022
(86) PCT No.: PCT/EP2022/077386

§ 371 Date: Mar. 25, 2024

Related U.S. Application Data
(60) Provisional application No. 63/250,455, filed on Sep. 30, 2021.

(51) Int. Cl. C12Q 1/6837

(52) U.S. Cl. CPC C12Q 1/6837

(57) ABSTRACT

The invention relates to methods of preventing renaturation of single-stranded nucleic acid libraries during storage, the method comprising using blocking oligonucleotides substantially complementary to adaptor sequences in the nucleic acid library.

### FIELD OF THE INVENTION

The invention relates to methods of preventing renaturation of single-stranded nucleic acid template libraries.

### BACKGROUND OF THE INVENTION

The detection of analytes such as nucleic acid sequences that are present in a biological sample has been used as a method for identifying and classifying microorganisms, diagnosing infectious diseases, detecting and characterizing genetic abnormalities, identifying genetic changes associated with cancer, studying genetic susceptibility to disease, and measuring response to various types of treatment. A common technique for detecting analytes such as nucleic acid sequences in a biological sample is nucleic acid sequencing.

Advances in the study of biological molecules have been led, in part, by improvement in technologies used to characterise the molecules or their biological reactions. In particular, the study of the nucleic acids DNA and RNA has benefited from developing technologies used for sequence analysis.

Methods of nucleic acid amplification which allow amplification products to be immobilised on a solid support in order to form arrays comprised of clusters or “colonies” formed from a plurality of identical immobilised polynucleotide strands and a plurality of identical immobilised complementary strands are known. The nucleic acid molecules present in DNA colonies on the clustered arrays prepared according to these methods can provide templates for sequencing reactions.

One method for sequencing a polynucleotide template involves performing multiple extension reactions using a DNA polymerase to successively incorporate labelled nucleotides to a template strand. In such a “sequencing by synthesis” reaction a new nucleotide strand base-paired to the template strand is built up in the 5′ to 3′ direction by successive incorporation of individual nucleotides complementary to the template strand.

### SUMMARY OF THE INVENTION

In one aspect of the invention there is provided a method to prevent or minimise renaturation of single stranded template libraries, comprising use of at least one blocking oligonucleotide substantially complementary to a part of an adaptor sequence on a single stranded template, wherein the blocking oligonucleotide hybridises to the adaptor sequence at a first temperature, wherein said first temperature is a temperature at which the template library is stored; and dissociates from the adaptor sequence at a second temperature, wherein said second temperature is the template seeding hybridisation temperature.

In another aspect of the invention there is provided a method of storing a single-stranded template library, the method comprising providing a denatured single-stranded template library and using at least one blocking oligonucleotide to prevent renaturation of the template library during storage, wherein the blocking oligonucleotide is substantially complementary to a part of an adaptor sequence on a single stranded template.

In a further aspect of the invention there is provided a method of seeding a double-stranded template library onto a solid substrate, the method comprising (i) denaturing the double-stranded template library to form a single-stranded template library; (ii) applying at least one blocking oligonucleotide to prevent renaturation of the single-stranded template library; (iii) maintaining the library at a temperature between 2° and 40° C., preferably between 2° and 35° C., more preferably between 25 and 35° C., even more preferably between 3° and 35° C., and typically about or at 35° C., until required for seeding; (iii) increasing the temperature to above 40° C., preferably 40 and 60° C., more preferably between 45 and 60° C., even more preferably between 5° and 60° C., yet even more preferably between 5° and 55° C., and typically about or at 50° C., to dissociate the at least one blocking oligonucleotide and (iv) hybridising the single stranded template library to an amplification primer immobilised onto the solid substrate, wherein the blocking oligonucleotide is substantially complementary to a part of an adaptor sequence on a single stranded template.

In a further aspect of the invention there is provided a hybridisation buffer, wherein the hybridisation buffer comprises a neutralisation agent and at least one blocking oligonucleotide; wherein said blocking oligonucleotide is substantially complementary to at least one part of an adaptor sequence on a single-stranded template library strand, wherein said blocking oligonucleotide is configured to hybridise to the adaptor sequence at a first temperature, wherein said first temperature is a temperature at which the template library is stored; and wherein said blocking oligonucleotide is also configured to dissociate from the adaptor sequence at a second temperature, wherein said second temperature is the template seeding hybridisation temperature.

In another aspect of the invention there is provided a P5′ blocking oligonucleotide comprising a sequence selected from SEQ ID NO: 5, 6 and 7 or a variant thereof, wherein the variant has at least 80% sequence identity to SEQ ID NO: 5, 6 and 7.

In another aspect of the invention there is provided a P7′ blocking oligonucleotide comprising a sequence selected from SEQ ID NO: 8, 9 and 10 or a variant thereof, wherein the variant has at least 80% sequence identity to SEQ ID NO: 8, 9 and 10.

In another aspect of the invention there is provided a blocked template library, wherein the template library comprises a plurality of single stranded template sequences and a plurality of blocking oligonucleotides, wherein the blocking oligonucleotides are hybridised to at least one part of an adaptor sequence, preferably the P5′ and/or P7′ primer binding sequence of an adaptor sequence, wherein the sequence of the P5′ primer-binding sequence comprises SEQ ID NO: 3 or a variant thereof and the sequence of the P7′ primer-binding sequence comprises SEQ ID NO: 4 or a variant thereof.

In another aspect of the invention there is provided a nucleic acid construct comprising at least one nucleic acid selected from SEQ ID NO: 5, 6, 7, 8, 9 and 10 or a variant thereof, wherein the variant has at least 80% sequence identity to SEQ ID NO: 5, 6, 7, 8, 9 and 10 and wherein the nucleic acid sequence is operably linked to a regulatory sequence, preferably a promoter.

### DESCRIPTION OF THE DRAWINGS

The invention is further described in the following non-limiting figures:

FIG. 1 is a schematic of renaturation of a single-stranded library in the absence of blocking oligonucleotides of the invention.

FIG. 2 is a schematic showing (A) the use of P5 blockers and (B) both P5 and P7 blocking oligonucleotides.

FIG. 3 shows the design of the P5 blocking oligonucleotides.

FIG. 4 shows the design of the P7 blocking oligonucleotides.

FIG. 5 shows the effect of different concentrations of a pool of blocking oligonucleotides on the hybridisation efficiency of a template library.

FIG. 6 shows the effect of blocking oligonucleotides on usable yield, clusters PF and occupancy.

### DETAILED DESCRIPTION OF THE INVENTION

The following features apply to all aspects of the invention.

Sequencing generally comprises four fundamental steps: 1) library preparation to form a plurality of template molecules available for sequencing; 2) cluster generation to form an array of amplified single template molecules on a solid support; 3) sequencing the cluster array; and 4) data analysis to determine the target sequence.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by PCR amplification and sequencing. The original sample DNA fragments are referred to as “inserts.” Alternatively “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size-fractionated prior to modification with the adaptor sequences.

As used herein an “adapter” sequence comprises a short sequence-specific oligonucleotide that is ligated to the 5′ and 3′ ends of each DNA (or RNA) fragment in a sequencing library as part of library preparation. The adaptor sequence may further comprise non-peptide linkers.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

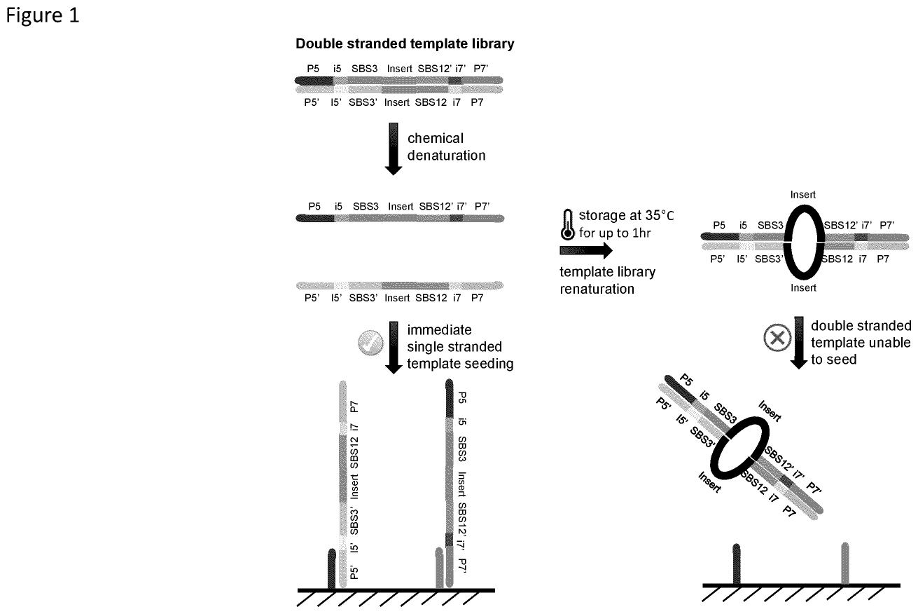

An example of a typical single-stranded nucleic acid template is shown in FIG. 1. In one embodiment, the template comprises, in the 5′ to 3′ direction, a first primer-binding sequence (e.g. P5), an index sequence (e.g. i5), a first sequencing binding site (e.g. SBS3), an insert, a second sequencing binding site (e.g. SBS12), a second index sequence (e.g. i7) and a second primer-binding sequence (e.g. P7). In another embodiment, the template comprises, in the 3′ to 5′ direction, a first primer-binding site (e.g. P5′, which is complementary to P5), an index sequence (e.g. 5′, which is complementary to 15), a first sequencing binding site (e.g. SBS3′ which is complementary to SBS3), an insert, a second sequencing binding site (e.g. SBS12′, which is complementary to SBS12), a second index sequence (e.g. i7′, which is complementary to 17) and a second primer-binding sequence (e.g. P7′, which is complementary to P7). Either template is referred to herein as a “template strand” or “a single stranded template”. Both template strands annealed together as shown in FIG. 1, is referred to herein as “a double stranded template”. The combination of a primer-binding sequence, an index sequence and a sequencing binding site is referred to herein as an adaptor sequence, and a single insert is flanked by a 5′ adaptor sequence and a 3′ adaptor sequence. The first primer-binding sequence may also comprise a sequencing primer for the index read (e.g. 15).

The P5′ and P7′ primer-binding sequences are complementary to short primer sequences (or lawn primers) present on the surface of the flow cells. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers will typically be around 20-40 nucleotides in length, although, in embodiments, the invention is not limited to sequences of this length. In embodiments, the precise identity of the amplification primers may vary, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analyzed in a single run, without drastically increasing run cost or run time. Examples of tag sequences are found in WO05068656, whose contents are incorporated herein by reference in their entirety. The tag can be read at the end of the first read, or equally at the end of the second read, for example using a sequencing primer complementary to a P7 sequence. The invention is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example U.S. 60/899,221. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The DNA polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g. SBS3′) followed by synthesis and sequencing of the complementary strand. This leads to the sequencing of the insert. In a second step, an index sequencing primer (e.g. 7 sequencing primer) binds to a second sequencing binding site (e.g. SBS12) leading to synthesis and sequencing of the index sequence (e.g. sequencing of the i7 primer). The second sequencing read may comprise binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. 7 sequencing primer) binds to a second sequencing binding site (e.g. SBS12′) leading to synthesis and sequencing of the insert in the reverse direction.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one embodiment, chemical denaturation is used, for example, by treatment with aqueous sodium hydroxide or formamide-based reagents (e.g. LDR, supplied by Illumina).

Following denaturation, a single-stranded template library can be contacted in free solution onto a solid support comprising surface capture moieties (for example P5 and P7 primers). This solid support is typically a flowcell, although in alternative embodiments, seeding and clustering can be conducted off-flowcell using, for example, microbeads or the like.

As used herein, the term “solid support” refers to a rigid substrate that is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fibre bundles, and polymers. A particularly useful material is glass.

Briefly, following attachment of the P5 and P7 primers, the solid support is contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, 5×SSC at 50° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. Further rounds of amplification (analogous to a standard PCR reaction) lead to the formation of clusters or colonies of template molecules bound to the solid support. Other amplification procedures may be used, and will be known to the skilled person. For example, amplification may be isothermal amplification using a strand displacement polymerase; or may be exclusion amplification as described in WO 2013/188582.

While a chemically denatured library that is kept cool and loaded quickly onto the flow cell remains sufficiently single stranded (ss) to enable efficient seeding, denatured libraries that are held at warmer conditions, e.g. 35° C., for prolonged periods of time, e.g. one hour or longer, will begin to re-anneal. This process is called renaturation. When such a renaturated library is used to seed a solid substrate (e.g. flow cell), the template is unable to bind the immobilised primers leading to a significant loss in seeding or hybridisation efficiency and consequently a significant reduction in overall sequencing efficiency. It is known that a ss-library demonstrates a reduction in effectiveness due to re-annealing of the ss-templates. Non-productive template reannealing is both temperature dependent and concentration dependent. It decreases seeding efficiency and slows down seeding kinetics.

It has been found that blocking oligonucleotides (or blocking oligos) can be used in the step between denaturation of the double stranded template library and seeding onto the flow cell to prevent or otherwise minimise unwanted annealing between the template strands.

Accordingly, the present invention provides a method to prevent or minimise renaturation of single stranded template libraries, comprising use of at least one blocking oligonucleotide substantially complementary to a part of an adaptor sequence present on the single stranded template, wherein the blocking oligonucleotide hybridises to the adaptor sequence at a first temperature, wherein said first temperature is a temperature at which the template library is stored; and dissociates from the adaptor sequence at a second temperature, wherein said second temperature is the template seeding hybridisation temperature.

By “hybridises” is meant that at least 60%, at least 70%, at least 80%, at least 90% of the adaptor sequences hybridise to a blocking oligonucleotide.

By “dissociates” is meant that at least 50%, at least 60%, at least 70%, at least 80%, at least 90% of the previously hybridised blocking oligonucleotide dissociate from the adaptor sequences.

Accordingly, the present invention provides a method to prevent or minimise renaturation of single stranded template libraries, comprising use of at least one blocking oligonucleotide substantially complementary to a part of an adaptor sequence present on the single stranded template, wherein the blocking oligonucleotide is hybridised to the adaptor sequence at a first temperature, wherein said first temperature is a temperature at which the template library is stored; and is dissociated from the adaptor sequence at a second temperature, wherein said second temperature is the template seeding hybridisation temperature.

By “hybridised” is meant that at least 60%, at least 70%, at least 80%, at least 90% of the adaptor sequences are hybridised to one or more blocking oligonucleotides.

By “dissociated” is meant that at least 50%, at least 60%, at least 70%, at least 80%, at least 90% of the previously hybridised blocking oligonucleotides are dissociated from the adaptor sequences.

By “prevent” is meant that no or no detectable amount of re-annealing (or renaturation) occurs. Again, this could be determined by measuring hybridisation efficiency. When using the oligo blockers, if the hybridisation efficiency in a denatured library that has been stored at warmer temperatures and/or for long periods of time is substantially the same as the hybridisation efficiency of a library that is denatured and immediately seeded and/or kept a low temperatures, it can be considered that the blocking oligos have prevented renaturation. By “minimise” is meant that the amount of renaturation is decreased compared to the level of renaturation of a template library where blocking oligonucleotides are not used. Again, this can be measured by assaying hybridisation efficiency.

By “renaturation” or re-annealing (such terms can be used interchangeably) means the re-forming of or a part of the complementary strands of the single-stranded templates into double-stranded templates. Typically, re-annealing will begin at the 5′ P5/3′ P5′ or 5′ P7/3′ P7′ ends of the template. As such, renaturation may be used to refer only to the renaturation at the 5′ P5/3′ P5′ or 5′ P7/3′ P7′ ends of the template. Alternatively, renaturation may occur over the whole sequence. As explained above, re-annealing of the single-stranded library prior to seeding on the flow cell will prevent or minimise the amount of template able to hybridise the immobilised P5 and P7 primers on the flow cell. Therefore, any renaturation that prevents or would prevent or reduce subsequent hybridisation of the template to the amplification primers is intended to fall within the scope of the invention. One way to measure the level of renaturation is to measure the amount of template hybridised to the flow cell—i.e. the hybridisation efficiency. As shown in Example 1, this can be determined as a % of hybridised template (compared to total amount of template) using the HybE assay.

As described above, each single stranded template may comprise a 5′ adaptor sequence comprising a P5 or P7 primer-binding sequence and a 3′ adaptor sequence comprising a P5′ or P7′ primer. Accordingly, in one embodiment, the method comprises use of at least one blocking oligonucleotide substantially complementary to at least one primer-binding sequence selected from a P5, P5′, P7 and P7′ primer or part thereof. Preferably, the at least one blocking oligonucleotide is substantially complementary to at least one primer-binding sequence selected from a P5′ or P7′ primer or part thereof. This is so that the blocking oligonucleotides do not substantially interact with the P5 and P7 primers that are typically immobilised on a standard flow cell. In an alternative embodiment, the method comprises the use of at least one blocking oligonucleotide substantially complementary to at least one index or at least one sequencing binding site or part thereof. In embodiments, by a “part thereof” is meant between 10 and 20 nucleotides, more preferably between 11 and 14 nucleotides, even more preferably between 12 and 13 nucleotides of, for example, the 5′ end, the 3′ end or anywhere between the 5′ and 3′ end of the primer-binding sequence.

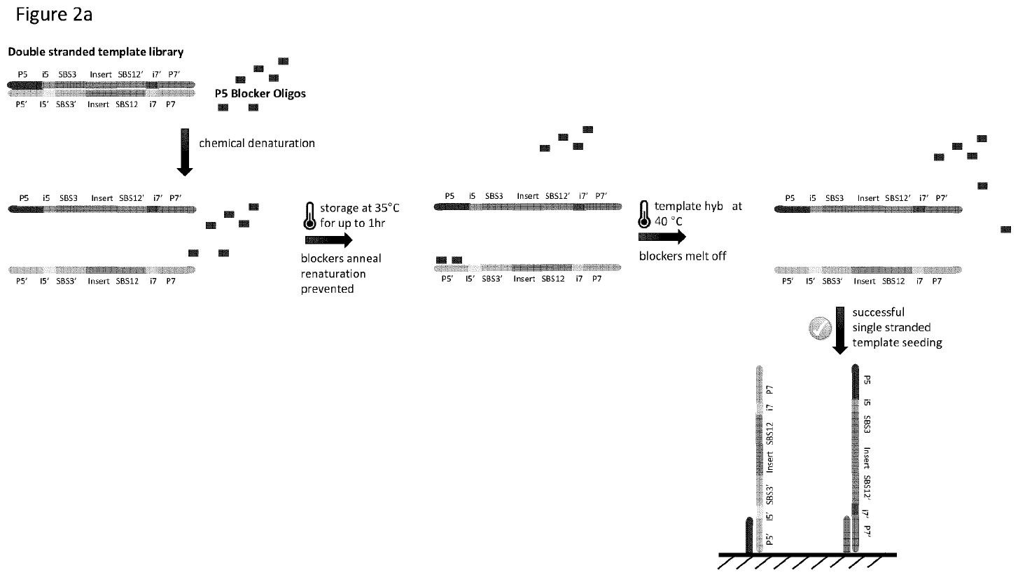

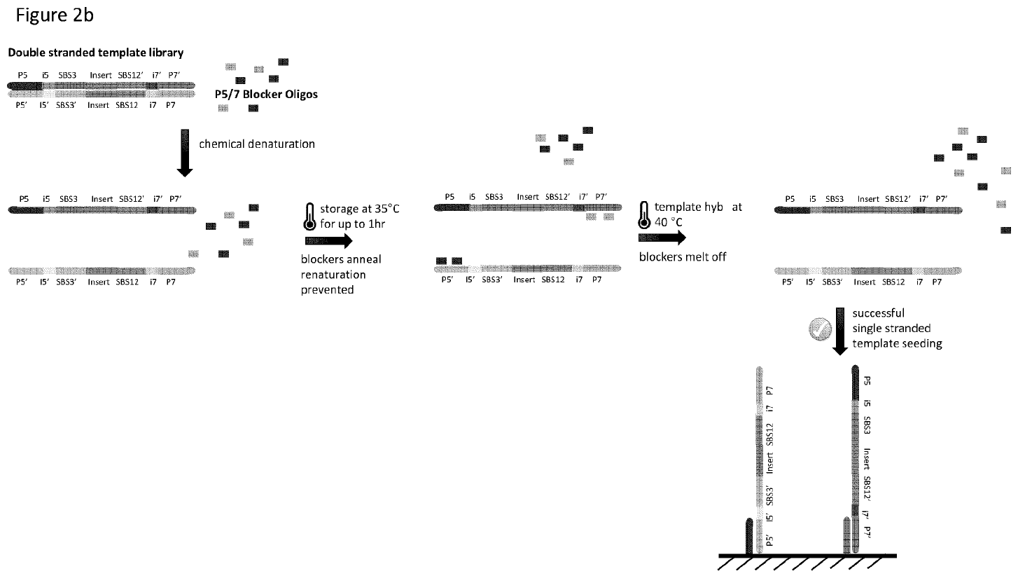

In one embodiment, the sequence of the P5 primer-binding sequence comprises SEQ ID NO: 1 or a variant thereof, the sequence of the P5′ adaptor comprises SEQ ID NO: 3 or a variant thereof, the sequence of the P7 adaptor comprises SEQ ID NO: 2 or a variant thereof and the sequence of the P7′ adaptor comprises SEQ ID NO: 4 or a variant thereof. As shown in FIG. 2, following denaturation of the double-stranded template library, blocking oligonucleotides to a P5′ or P7′ primer will bind the complementary sequences in P5 and P7 preventing the single-stranded template strands from re-annealing at the P5/P5′ and/or P7/P7′ primer sites. Advantageously, these blockers have also been shown not self-hybridise.

By “complementary” is meant that the blocking oligo has a sequence of nucleotides that can form a double-stranded structure by matching base-pairs with the adaptor or primer sequence or part thereof. By “substantially complementary” is meant that the blocking nucleotides has at least 85%, 90%, 95%, 98% or 99% overall sequence identical to the complementary sequence.

The term “variant” as used herein with reference to any of the sequence recited herein refers to a variant nucleic acid that is substantially identical, i.e. has only some sequence variations, for example to the non-variant sequence. In one embodiment, a variant has at least 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid sequence.

In one embodiment, the blocking oligonucleotide hybridises to the single stranded template at or around a first storage temperature or temperature range of the template library and dissociates from the single stranded template at or around the second template seeding hybridisation temperature or temperature range. By “a first temperature” or a “first storage temperature” is meant a temperature at which the template library is held during a sequencing run (e.g. after denaturation) for a period of time, typically an hour or above. The storage temperature can be between 2° and 40° C., preferably between 2° and 35° C., more preferably between 25 and 35° C., even more preferably between 3° and 35° C., and typically is about or is 35° C. In one embodiment, the storage temperature may be an initial temperature between 2° and 40° C., preferably between 2° and 35° C., more preferably between 2° and 30° C., even more preferably between 2° and 25° C., and typically is about or is 20° C.; and may be followed by a subsequent temperature different from the initial temperature between 2° and 40° C., preferably between 2° and 35° C., more preferably between 25 and 35° C., even more preferably between 3° and 35° C., and typically is about or is 35° C. In one example, the library may be held at more than one hour at a range between 2° and 40° C., such as an hour at around 20° C. and an hour at around 35° C.

By “a second temperature” or “a second template seeding hybridisation temperature” is meant the temperature used to seed (i.e. hybridise) the template strand to a primer sequence, such as an immobilised P5 or P7 primer on the surface of a flow cell (which subsequently leads to amplification of the primer as the first step in the sequencing process). The second temperature can be between 4° and 60° C., preferably between 45 and 60° C., more preferably between 5° and 60° C., even more preferably between 5° and 55° C., and typically is about or is 50° C.

The conditions under which two sequences will hybridise and denature—be that the blocking oligonucleotide to an adaptor sequence (or part thereof such as P5′ and P7′) in the template strand, or the template strand to a primer sequence, depends largely on the melting temperature (Tm) of that sequence. Above the Tm, the hybridised strands will be mostly or all single-stranded; below the Tm, the hybridised strands will be mostly double-stranded. For DNA, the Tm depends primarily on its G+C content, as well as the length of the sequence (e.g. the length of the blocking oligonucleotide or the primer).

The Tm can be calculated using the IDT OligoAnalyzer Tool version 3.1 (available at https://eu.idtdna.com/pages/tools/oligoanalyzer).

Accordingly, it has been found that using blocking oligonucleotides with a Tm that is below (e.g. at least about 5, 6, 7, 8, 9 or 10° C. below, preferably at least about 5° C. below) the temperature used to hybridise a template strand to its amplification primer, the blocking oligonucleotides can be used to prevent or minimise renaturation of a single-stranded template while the library is held at a storage temperature that is below the seeding hybridisation temperature. Increasing the temperature to or above the Tm of the blocking oligonucleotide will cause the blocking oligonucleotides to dissociate or “melt-off” the template strands, allowing the templates to hybridise to the amplification primers. This is shown in FIG. 2.

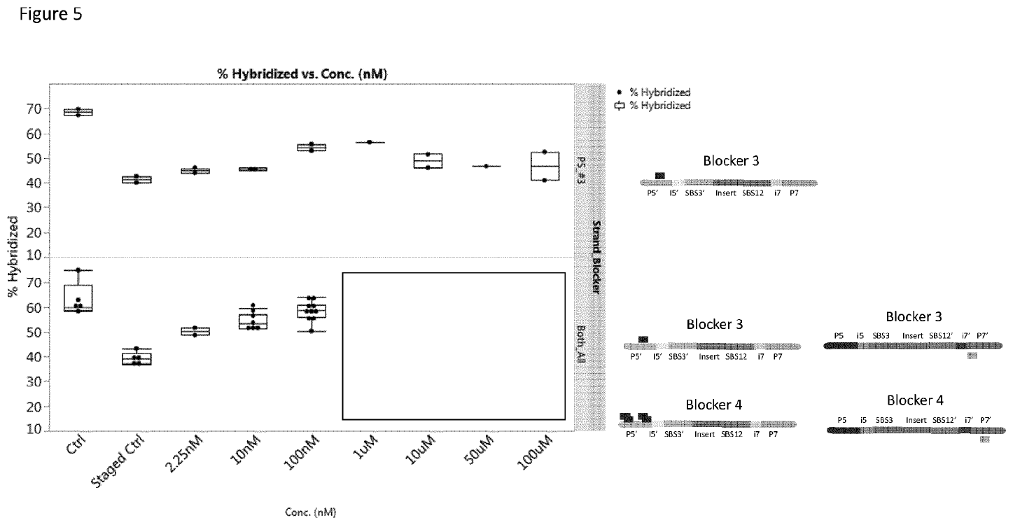

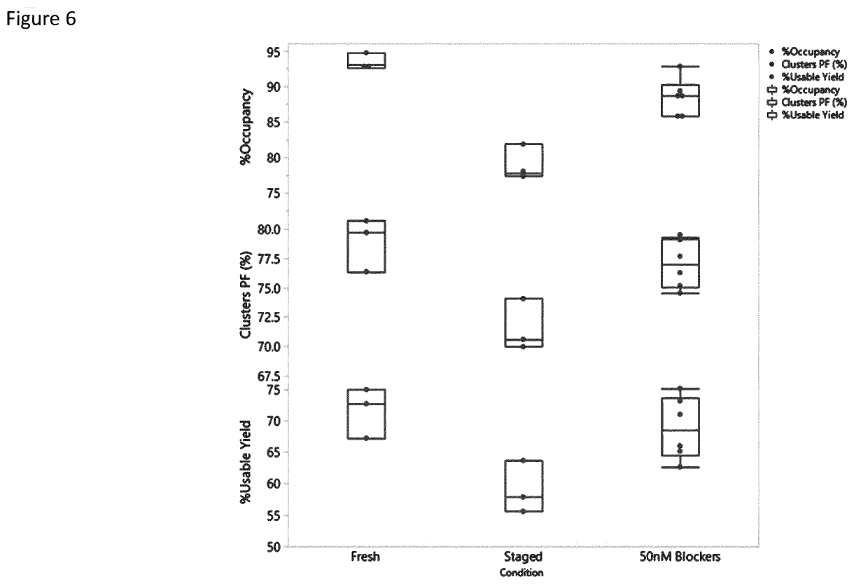

In one embodiment, at least one blocking oligonucleotide substantially complementary to a part of a P5′ adaptor sequence is used with at least one blocking oligonucleotide substantially complementary to a part of a P7′ adaptor sequence. This is shown in FIG. 2b. As shown in FIGS. 5 and 6, the use of blocking oligonucleotides to both P5′ and P7′ improves % hybridisation efficiency, % useable yield, % clusters PF and % occupancy compared to levels where no blocking oligonucleotides were used. In fact, as shown in FIGS. 5 and 6, the % hybridisation efficiency, % useable yield, % clusters PF and % occupancy were very similar to those observed when a denatured library was immediately seeded (i.e. providing no opportunity for renaturation to occur).

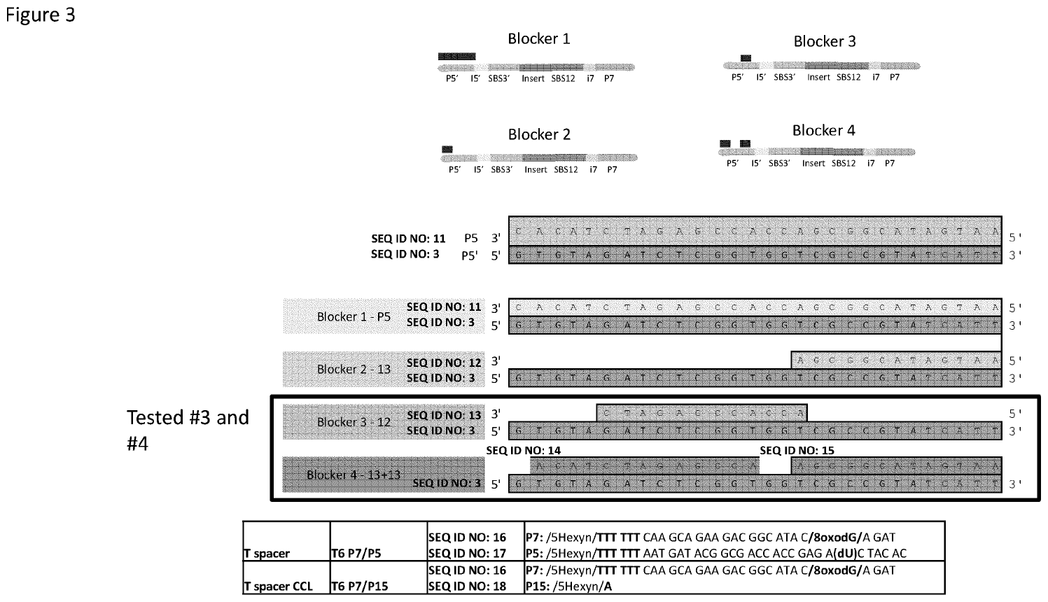

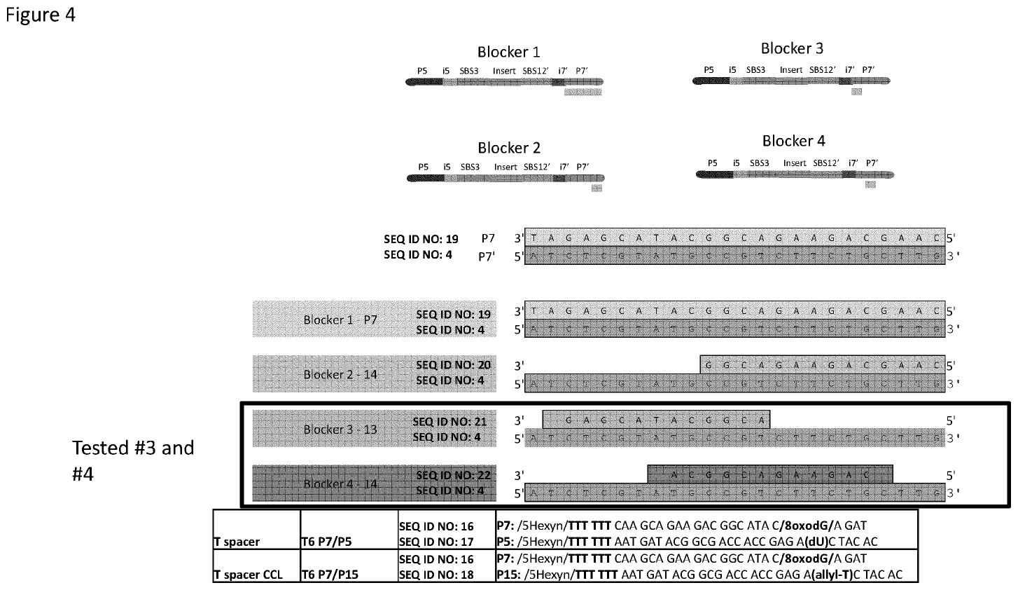

In a further embodiment, the method comprises use of at least two or more blocking oligonucleotides substantially complementary to at least two (different regions (or parts)) of a P5′ primer-binding sequence and/or two or more blocking oligonucleotides substantially complementary to different regions of a P7′ primer-binding sequence. For example, the blocking oligonucleotides may be complementary to the 5′ end, the 3′ end or anywhere in between the P5′ or P7′ primer binding sequence. This is shown in FIGS. 3 and 4.

In a further embodiment, a plurality of blocking oligonucleotides are used to create a pool of blocking oligonucleotides, where the pool comprises a plurality of blocking oligonucleotides to both P5′ and P7′ primer-binding sequences, and where the P5′ and/or P7′ blocking oligonucleotides are substantially complementary to multiple regions on the P5′ and/or P7′ primer binding sequence.

In one embodiment, the at least one blocking oligonucleotide has a length between 10 and 20 nucleotides. In another embodiment, the blocking oligonucleotide has a length between 11 and 14 nucleotides. In a further embodiment, the at least one blocking oligonucleotide is either 12 or 13 nucleotides in length.

In another embodiment, the blocking oligonucleotide has a melting temperature (Tm) between 3° and 45° C., preferably between 35 and 43° C. In one embodiment, the melting temperature is about 37° C. or about 38° C. or about 39° C., or about 40° C., or about 41° C. or about 42° C.

In one embodiment, the P5′ blocking oligonucleotide comprises a sequence from SEQ ID NO: 5, 6 or 7 or a variant thereof, wherein the variant has at least 80% sequence identity to SEQ ID NO: 5, 6 or 7. More preferably, the variant has at least 86%, 87%, 88%, 89%, 90%, 91%, 92%, 93%, 94%, 95%, 96%, 97%, 98%, or at least 99% overall sequence identity to SEQ ID NO: 5, 6 or 7. In another embodiment, the P5′ blocking oligonucleotide consists of a sequence selected from SEQ ID NO: 5, 6 or 7.

In another embodiment, the P7′ blocking oligonucleotide comprises a sequence selected from SEQ ID NO: 8, 9 or 10 or a variant thereof, wherein the variant has at least 80% sequence identity to SEQ ID NO: 8, 9 or 10. More preferably, the variant has at least 86%, 87%, 88%, 89%, 90%, 91%, 92%, 93%, 94%, 95%, 96%, 97%, 98%, or at least 99% overall sequence identity to SEQ ID NO: 8, 9 or 10. In another embodiment, the P7′ blocking oligonucleotide consists of a sequence selected from SEQ ID NO: 8, 9 or 10.

In one embodiment, a pool of blocking oligonucleotides may be used. For example, the pool of blocking oligonucleotides may comprise two or more types of blocking oligonucleotides, preferably two to five types of blocking oligonucleotides, more preferably five types of blocking oligonucleotides.

In one embodiment, the pool of blocking oligonucleotides may comprise a first blocking oligonucleotide and a second blocking nucleotide. In a preferred embodiment, the first blocking oligonucleotide may be a P5′ blocking oligonucleotide (e.g. comprising SEQ ID NO: 5, 6 or 7, preferably SEQ ID NO: 6) and the second blocking oligonucleotide may be a P5′ blocking oligonucleotide (e.g. comprising SEQ ID NO: 5, 6 or 7) or a P7′ blocking oligonucleotide (e.g. comprising SEQ ID NO: 8, 9 or 10). In another preferred embodiment, the first blocking oligonucleotide may be a P7′ blocking oligonucleotide (e.g. comprising SEQ ID NO: 8, 9 or 10) and the second blocking oligonucleotide may be a P5′ blocking oligonucleotide (e.g. comprising SEQ ID NO: 5, 6 or 7) or a P7′ blocking oligonucleotide (e.g. comprising SEQ ID NO: 8, 9 or 10).

In one embodiment, the pool of blocking oligonucleotides may further comprise a third blocking nucleotide. In one embodiment, the third blocking oligonucleotide may be a P5′ blocking oligonucleotide (e.g. comprising SEQ ID NO: 5, 6 or 7) or a P7′ blocking oligonucleotide (e.g. comprising SEQ ID NO: 8, 9 or 10).

In one embodiment, the pool of blocking oligonucleotides may further comprise a fourth blocking nucleotide. In one embodiment, the fourth blocking oligonucleotide may be a P5′ blocking oligonucleotide (e.g. comprising SEQ ID NO: 5, 6 or 7) or a P7′ blocking oligonucleotide (e.g. comprising SEQ ID NO: 8, 9 or 10).

In one embodiment, the pool of blocking oligonucleotides may further comprise a fifth blocking nucleotide. In one embodiment, the fifth blocking oligonucleotide may be a P5′ blocking oligonucleotide (e.g. comprising SEQ ID NO: 5, 6 or 7) or a P7′ blocking oligonucleotide (e.g. comprising SEQ ID NO: 8, 9 or 10).

In one embodiment, the pool of blocking oligonucleotides may comprise three P5′ blocking oligonucleotides (e.g. each independently comprising SEQ ID NO: 5, 6 or 7) and two P7′ blocking oligonucleotides (e.g. each independently comprising SEQ ID NO: 8, 9 or 10). Preferably, the pool of blocking oligonucleotides may comprise a first blocking oligonucleotide comprising SEQ ID NO: 6 or a variant thereof, a second blocking oligonucleotide comprising SEQ ID NO: 5 or a variant thereof, a third blocking oligonucleotide comprising SEQ ID NO: 7 or a variant thereof, a fourth blocking oligonucleotide comprising SEQ ID NO: 9 or a variant thereof, and a fifth blocking oligonucleotide comprising SEQ ID NO: 10 or a variant thereof.

In another embodiment, the blocking oligonucleotides comprise a modified 3′ nucleotide, wherein the modification prevents extension of the oligonucleotide by a DNA polymerase. For example, the modified nucleotide comprises a phosphate group, e.g. a phosphate group attached to the 3′ end. This prevents amplification of the blocking oligonucleotide by a DNA polymerase.

In one embodiment, the single stranded template library is formed following denaturation of a double-stranded template library, and wherein the at least one blocking oligonucleotide is added to the single stranded template library following denaturation.

In a further embodiment, the blocking oligonucleotides are added at a concentration between 2 and 500 nM, more preferably between 10 and 100 nM. In one embodiment, the blocking oligonucleotides are added at 10 or 100 mM.

In another aspect of the invention, there is provided a method of storing a single-stranded template library, the method comprising providing a denatured single-stranded template library and using at least one blocking oligonucleotide of the invention to prevent renaturation of the template library. In one embodiment, the library may be stored at between 2° and 40° C., preferably between 2° and 35° C., more preferably between 25 and 35° C., even more preferably between 3° and 35° C., and typically is about or is 35° C., for at least one hour. In one embodiment, the library may be stored at an initial temperature between 2° and 40° C., preferably between 2° and 35° C., more preferably between 2° and 30° C., even more preferably between 2° and 25° C., and typically about or at 20° C., for at least one hour; and then at a subsequent temperature different from the initial temperature between 2° and 40° C., preferably between 2° and 35° C., more preferably between 25 and 35° C., even more preferably between 3° and 35° C., and typically about or at 35° C., for at least one hour.

In an embodiment, the methods of the present invention prevent renaturation of the template library over a time period of at least 90 minutes, or at least 2, 3, 4, 5, 6, 7, 8, 9, 10, 11 or 12 hours. In a further embodiment, the methods of the present invention minimise renaturation of the template library over a time period of at least 90 minutes, or at least 2, 3, 4, 5, 6, 7, 8, 9, 10, 11 or 12 hours. In embodiments according to the present invention for the time periods herein, the template library may be stored a temperature of below 40° C., 39° C., 38° C., 37° C., 36° C., 35° C., 34° C., 33° C., 32° C., 31° C. or 30° C.

In another aspect of the invention, there is provided a method of seeding a double-stranded template library onto a solid substrate comprising at least one immobilised primer, the method comprising (i) denaturing the double-stranded library to form a single-stranded library; (ii) applying at least one blocking oligonucleotide of the invention to prevent renaturation of the template library; (iii) maintaining the library at a temperature between 2° and 40° C., preferably between 2° and 35° C., more preferably between 25 and 35° C., even more preferably between 3° and 35° C., and typically about or at 35° C., until required for seeding; (iii) increasing the temperature to above 40° C., preferably 40 and 60° C., more preferably between 45 and 60° C., even more preferably between 5° and 60° C., yet even more preferably between 5° and 55° C., and typically about or at 50° C., to melt off the at least one blocking oligonucleotide and (iv) hybridising the single stranded template library to the immobilised primers. In embodiments, the blocking oligonucleotides according to the present invention are added before denaturation in step (i).

In another aspect of the invention, there is provided a hybridisation buffer, wherein the hybridisation buffer comprises a neutralisation agent and at least one blocking oligonucleotide of the invention.

In another aspect of the invention there is provided a P5′ blocking oligonucleotide comprising a sequence selected from SEQ ID NO: 5, 6 and 7 or a variant thereof, wherein the variant has at least 80% sequence identity to SEQ ID NO: 5, 6 and 7. In a further aspect, there is provided a P7′ blocking oligonucleotide comprising a sequence selected from SEQ ID NO: 8, 9 and 10 or a variant thereof, wherein the variant has at least 80% sequence identity to SEQ ID NO: 8, 9 and 10. Alternatively, the variant may have at least 85%, 90%, 95%, 98% or 99% overall sequence identical to any of the recited sequences.

In another aspect of the invention, there is provided a blocked single-stranded template library, wherein the template library comprises a plurality of single stranded template sequences and a plurality of blocking oligonucleotides of the invention, wherein the blocking oligonucleotides are substantially complementary to a part of an adaptor sequence on the single-stranded template.

As such, the blocking oligonucleotides hybridise to the adaptor sequence preventing the single-stranded template from re-annealing. In this way, the template library can be considered “blocked”.

The invention is now described in the following non-limiting examples:

### Example 1

Blocker oligos were ordered from IDT as shown in Table 1. All oligos were ordered with a 3′ phosphate block. Oligos were resuspended to 100 uM in buffer. Equal volumes of each oligo were combined and diluted to 1, 5, and 10 uM to form the Blocker mixes to be spiked into samples, depending on intended final concentration of the blockers.

TABLE 1
Blocker Sequences and Properties

| Blocker | | | | | |
|---|---|---|---|---|---|
| characteristic | P5_3 | P5_4a | P5_4b | P7_3 | P7_4 |
| Primer Strand | P5 | P5 | P5 | P7 | P7 |
| Tm (C.) | 40.9 | 39.1 | 41.6 | 42.2 | 42.4 |
| Sequence | ACC ACC GAG | ACC GAG ATC | AAT GAT ACG | ACG GCA TAC | CAG AAG ACG |
| | ATC | TAC A | GCG A | GAG | GCA |
| Length | 12 | 13 | 13 | 12 | 12 |
| % GC content | 58.3 | 46.2 | 46.2 | 58.3 | 58.3 |
| Block | 3′ Phos | 3′ Phos | 3′ Phos | 3′ Phos | 3′ Phos |
| Purification | Desalting | Desalting | Desalting | Desalting | Desalting |

For testing of staged samples±Blockers, template was denatured in bulk and diluted with HT1 (hybridization buffer) (Illumina). The sample was aliquoted into separate tubes and Blockers were added to ‘+ Blocker’ condition tubes at a range of concentrations depending on the test. The staging protocol was as follows: 1 hour at 20° C. followed by 1 hour at 35° C. This was to simulate the worst case conditions in practice.

Samples were analyzed by either the hybridization efficiency assay (HybE) or via sequencing. The HybE assay is cBot-based and relies on qPCR to quantify the amount of DNA hybridized to the flowcell relative to the starting amount (i.e. % hybridized). It works by performing hybridization and washing away DNA that is not captured onto the flowcell. The flowcell is then heated to denature DNA, and the sample is pumped back into a collection tube. qPCR is performed on both the starting sample and the sample captured after hybridization.

### Example 2

Following the method recited in Example 1, Blocker 3-P5 (SEQ ID NO: 6) was analysed for its ability to prevent renaturation of a single stranded DNA library. In addition, a pool of P5 and P7 blocking oligonucleotides were analysed for their ability to prevent renaturation of a single stranded DNA library. The pool of P5 and P7 blocking oligos was made up of Blocker 3-P5 (SEQ ID NO: 6), Blocker 4-P5 (SEQ ID NO: 5 and SEQ ID NO: 7), Blocker 3-P7 (SEQ ID NO: 9) and Blocker 4-P7 (SEQ ID NO: 10), all of which target different regions on P5′ and P7′. To determine whether the single blocker or the pool of blocking oligos prevented (or minimised) renaturation, the hybridisation efficiency of the template DNA library was assessed as described above.

As shown in FIG. 5 under control conditions—that is, where the denatured library is kept cool and loaded quickly onto the flow cell, the amount of template DNA hybridised to the flow cell is around 60-70%.

In the single blocker experiment (FIG. 5, top), under staged conditions—that is, where the denatured library is held at 1 hour at 35° C., the amount of template DNA that subsequently hybridised to the flow cell dropped to around 40%. Addition of 2.25 nM of Blocker 3-P5 increased the level of hybridisation to just under 50%, whilst addition of 100 nM to 1 uM increased the level of hybridisation to just under 60%. High levels (relative to staged control) can also be seen at 10 uM to 100 uM concentrations. This indicates that a single blocker oligo can be used to reduce/minimise the level of renaturation.

In the pool of blockers experiment (FIG. 5, bottom), under staged conditions, the amount of template DNA that subsequently hybridised to the flow cell dropped to around 40%—a decrease of approx. 65% compared to the level of hybridisation in the control. Addition of 2.25 nM of the pool of blocking oligonucleotides increased the level of subsequent template hybridisation to over 50%. However, addition of 10 nM or 100 nM of the pool of blocking oligonucleotides increased levels of hybridisation under staged conditions to just under 60%. This is similar to the levels of hybridisation observed under control conditions (i.e. no staging) and demonstrates that the blocking oligos can be used to prevent renaturation and increase hybridisation efficiency of a single-stranded template library when held in warm conditions for long periods of time.

### Example 3

Again following the method recited in Example 1, the addition of 50 nM of blockers was analysed for their effect on useable yield (calculated using [% PF−(% PF×% Dups)/100], and is an indication of the actual amount of usable data), clusters PF % (the % of clusters passing filters (PF) is an indication of signal purity from each cluster) and occupancy (the number of wells that contain a cluster, i.e. non-empty wells). The pool of blockers used was the same as Example 2 (i.e. Blocker 3-P5 (SEQ ID NO: 6), Blocker 4-P5 (SEQ ID NO: 5 and SEQ ID NO: 7), Blocker 3-P7 (SEQ ID NO: 9) and Blocker 4-P7 (SEQ ID NO: 10)). As shown in FIG. 6, the % of useable yield drops from between 70 and 75% in the control (fresh) to between 55 and 65% under staged conditions (1 hr at 20° C. and 1 hr at 35° C.), the % of clusters PF drops from between 80 and 75% in the control (to between 70 and 75% under the staged conditions and the % occupancy drops from between 90 and 95% in the control to between 75 and 83% under the staged conditions. However, when 50 nM of blocking oligos are added under the same staged conditions there is an increase in all of % useable yield (to between 65 and 75%), an increase of % of clusters PF (to between 75 and 80%) and an increase in % occupancy (to be between 85 and 90%)—all of which are very similar to the levels seen under control conditions. Again, this example demonstrates that blocking oligos can be used to prevent renaturation of a single-stranded template library, even when held in “worse case” conditions—1 hr at 20° C. and 1 hr at 35° C.

1-31. (canceled)

32. A method of increasing hybridisation efficiency when seeding a double-stranded template library onto a solid substrate, the method comprising (i) denaturing the double-stranded template library to form a single-stranded template library; (ii) applying at least one blocking oligonucleotide to prevent renaturation of the single-stranded template library; (iii) maintaining the library at a temperature between 2° and 40° C., until required for seeding; (iv) increasing the temperature to between 4° and 60° C. to dissociate the at least one blocking oligonucleotide; and (v) hybridising the single stranded template library to an amplification primer immobilised onto the solid substrate, wherein the blocking oligonucleotide is substantially complementary to a part of an adaptor sequence on a single stranded template; wherein hybridisation efficiency is increased compared to the hybridisation efficiency when at least one blocking oligonucleotide is not applied.

33. The method of claim 32, wherein the library is stored for a period of time of at least 1 hour at a temperature of between 2° and 35° C., more preferably between 25 and 35° C., even more preferably between 3° and 35° C., most preferably around 35° C.

34. The method of claim 32 or 33, wherein the method comprises increasing the temperature to between 45 and 60° C., even more preferably between 5° and 60° C., yet even more preferably between 5° and 55° C., and typically about or at 50° C., to dissociate the at least one blocking oligonucleotide.

35. The method of any one of claims 32 to 34, wherein the template comprises a 5′ adaptor sequence comprising a P5 or P7 primer-binding sequence and a 3′ adaptor sequence comprising a P5′ or P7′ primer-binding sequence, and wherein the at least one blocking oligonucleotide is substantially complementary to at least one part of at least one primer-binding sequence selected from a P5, P5′, P7 and P7′-binding sequence, preferably a P5′ and P7′-binding sequence, wherein the sequence of the P5 primer-binding sequence comprises SEQ ID NO: 1 or a variant thereof, the sequence of the P5′ primer-binding sequence comprises SEQ ID NO: 3 or a variant thereof, the sequence of the P7 primer-binding sequence comprises SEQ ID NO: 2 or a variant thereof and the sequence of the P7′ primer-binding sequence comprises SEQ ID NO: 4 or a variant thereof, wherein the variant has at least 80% sequence identity to SEQ ID NO: 1, 2, 3 or 4.

36. The method of any one of claims 32 to 35, wherein the method comprises use of at least one blocking oligonucleotide substantially complementary to at least one part of a P5′ primer-binding sequence (a P5′ blocking oligonucleotide) and/or at least one blocking oligonucleotide substantially complementary to at least one part of a P7′ primer-binding sequence (a P7′ blocking oligonucleotide).

37. The method of any one of claims 32 to 36, wherein the method comprises use of at least two blocking oligonucleotides substantially complementary to at least two different parts of a P5′ and/or P7′ primer-binding sequence.

38. The method of any one of claims 32 to 37, wherein the blocking oligonucleotide has a length between 11 and 14 nucleotides, and/or a melting temperature (Tm) between 3° and 45° C., wherein preferably the Tm is between 35 and 43° C.

39. The method of any one of claims 32 to 38, wherein the P5′ blocking oligonucleotide comprises a sequence from SEQ ID NO: 5, 6 or 7 or a variant thereof, wherein the variant has at least 80% sequence identity to SEQ ID NO: 5, 6 or 7.

40. The method of any one of claims 32 to 39, wherein the P7′ blocking oligonucleotide comprises a sequence selected from SEQ ID NO: 8, 9 or 10 or a variant thereof, wherein the variant has at least 80% sequence identity to SEQ ID NO: 8, 9 or 10.

41. The method of any one of claims 23 to 40, wherein the blocking oligonucleotides comprise at least one modified nucleotide, wherein preferably, the modified nucleotide is the 3′ terminal nucleotide of the blocking oligonucleotide, and the modified nucleotide comprises a 3′ phosphate group.

42. A hybridisation buffer, wherein the hybridisation buffer comprises a neutralisation agent and at least one blocking oligonucleotide; wherein said blocking oligonucleotide is substantially complementary to at least one part of an adaptor sequence on a single-stranded template library strand, wherein said blocking oligonucleotide is configured to hybridise to the adaptor sequence at a first temperature, wherein said first temperature is a temperature at which the template library is stored; and wherein the first storage temperature of the template library is between 2° and 40° C.; and wherein said blocking oligonucleotide is also configured to dissociate from the adaptor sequence at a second temperature, wherein said second temperature is the template seeding hybridisation temperature, and wherein the second temperature is between 4° and 60° C.

43. The hybridisation buffer of claim 42, wherein the at least one blocking oligonucleotide is substantially complementary to at least one part of a primer-binding sequence selected from a P5, P5′, P7 and P7′-binding sequence, preferably a P5′ and P7′-binding sequence, on the single-stranded template library strand, wherein the sequence of the P5 primer-binding sequence comprises SEQ ID NO: 1 or a variant thereof, the sequence of the P5′ primer-binding sequence comprises SEQ ID NO: 3 or a variant thereof, the sequence of the P7 primer-binding sequence comprises SEQ ID NO: 2 or a variant thereof and the sequence of the P7′ primer-binding sequence comprises SEQ ID NO: 4 or a variant thereof, wherein the variant has at least 80% sequence identity to SEQ ID NO: 1, 2, 3 or 4.

44. The hybridisation buffer of claim 43, wherein the buffer comprises at least one blocking oligonucleotide substantially complementary to at least one part of a P5′ primer-binding sequence (a P5′ blocking oligonucleotide) and/or at least one blocking oligonucleotide substantially complementary to at least one part of a P7′ primer-binding sequence (a P7′ blocking oligonucleotide).

45. The hybridisation buffer of any one of claims 42 to 44, wherein the blocking oligonucleotide has a length between 11 and 14 nucleotides, and/or a melting temperature between 3° and 45° C., preferably between 35 and 43° C.

46. The hybridisation buffer of any one of claims 43 to 45, wherein the P5′ blocking oligonucleotide comprises a sequence selected from SEQ ID NO: 5, 6 and 7 or a variant thereof, wherein the variant has at least 80% sequence identity to SEQ ID NO: 5, 6 and 7.

47. The hybridisation buffer of any of claims 43 to 45, wherein the P7′ blocking oligonucleotide comprises a sequence selected from SEQ ID NO: 8, 9 and 10 or a variant thereof, wherein the variant has at least 80% sequence identity to SEQ ID NO: 8, 9 and 10.

48. A P5′ blocking oligonucleotide comprising a sequence as defined in SEQ ID NO: 6 or a variant thereof, wherein the variant has at least 90% sequence identity to SEQ ID NO: 6.

49. A P7′ blocking oligonucleotide comprising a sequence selected from SEQ ID NO: 9 and 10 or a variant thereof, wherein the variant has at least 90% sequence identity to SEQ ID NO: 9 and 10.

50. A blocked template library, wherein the template library comprises a plurality of single stranded template sequences and a plurality of blocking oligonucleotides, wherein the blocking oligonucleotides are hybridised to at least one part of an adaptor sequence, preferably the P5′ and/or P7′ primer binding sequence of an adaptor sequence, wherein the sequence of the P5′ primer-binding sequence comprises SEQ ID NO: 3 or a variant thereof and the sequence of the P7′ primer-binding sequence comprises SEQ ID NO: 4 or a variant thereof, wherein said blocking oligonucleotide is configured to hybridise to the adaptor sequence at a first temperature, wherein said first temperature is a temperature at which the template library is stored; and wherein the first storage temperature of the template library is between 2° and 40° C.; and wherein said blocking oligonucleotide is also configured to dissociate from the adaptor sequence at a second temperature, wherein said second temperature is the template seeding hybridisation temperature, and wherein the second temperature is between 4° and 60° C.


SEQUENCE LISTING (22 sequences)

SEQ ID NO: 1 - DNA, 29 bp, synthetic construct
  source 1..29: mol_type = other DNA
aatgatacggcgaccaccgagatctacac

SEQ ID NO: 2 - DNA, 24 bp, synthetic construct
  source 1..24: mol_type = other DNA
caagcagaagacggcatacgagat

SEQ ID NO: 3 - DNA, 29 bp, synthetic construct
  source 1..29: mol_type = other DNA
gtgtagatctcggtggtcgccgtatcatt

SEQ ID NO: 4 - DNA, 24 bp, synthetic construct
  source 1..24: mol_type = other DNA
atctcgtatgccgtcttctgcttg

SEQ ID NO: 5 - DNA, 13 bp, synthetic construct
  source 1..13: mol_type = other DNA
aatgatacggcga

SEQ ID NO: 6 - DNA, 12 bp, synthetic construct
  source 1..12: mol_type = other DNA
accaccgagatc

SEQ ID NO: 7 - DNA, 13 bp, synthetic construct
  source 1..13: mol_type = other DNA
accgagatctaca

SEQ ID NO: 8 - DNA, 14 bp, synthetic construct
  source 1..14: mol_type = other DNA
caagcagaagacgg

SEQ ID NO: 9 - DNA, 12 bp, synthetic construct
  source 1..12: mol_type = other DNA
acggcatacgag

SEQ ID NO: 10 - DNA, 12 bp, synthetic construct
  source 1..12: mol_type = other DNA
cagaagacggca

SEQ ID NO: 11 - DNA, 29 bp, synthetic construct
  source 1..29: mol_type = other DNA
cacatctagagccaccagcggcatagtaa

SEQ ID NO: 12 - DNA, 13 bp, synthetic construct
  source 1..13: mol_type = other DNA
agcggcatagtaa

SEQ ID NO: 13 - DNA, 12 bp, synthetic construct
  source 1..12: mol_type = other DNA
ctagagccacca

SEQ ID NO: 14 - DNA, 13 bp, synthetic construct
  source 1..13: mol_type = other DNA
acatctagagcca

SEQ ID NO: 15 - DNA, 13 bp, synthetic construct
  source 1..13: mol_type = other DNA
agcggcatagtaa

SEQ ID NO: 16 - DNA, 30 bp, synthetic construct
  source 1..30: mol_type = other DNA
  modified_base 26: mod_base = OTHER; note = 8-Oxoguanine
ttttttcaagcagaagacggcatacgagat

SEQ ID NO: 17 - DNA, 35 bp, synthetic construct
  source 1..35: mol_type = other DNA
  modified_base 29: mod_base = OTHER; note = Uracil
ttttttaatgatacggcgaccaccgagatctacac

SEQ ID NO: 18 - DNA, 35 bp, synthetic construct; identical to SEQ ID NO: 17
  source 1..35: mol_type = other DNA
  modified_base 29: mod_base = OTHER; note = O-Allyl Thymine

SEQ ID NO: 19 - DNA, 24 bp, synthetic construct
  source 1..24: mol_type = other DNA
tagagcatacggcagaagacgaac

SEQ ID NO: 20 - DNA, 14 bp, synthetic construct
  source 1..14: mol_type = other DNA
ggcagaagacgaac

SEQ ID NO: 21 - DNA, 12 bp, synthetic construct
  source 1..12: mol_type = other DNA
cagcatacggca

SEQ ID NO: 22 - DNA, 12 bp, synthetic construct
  source 1..12: mol_type = other DNA
acggcagaagac